(19) United States
(12) Patent Application Publication
(10) Pub. No.: US 2022/0280063 A1
(43) Pub. Date: Sep. 8, 2022

(54) RESPIRATION DETECTION SYSTEM AND RESPIRATION DETECTION METHOD

(71) Applicant: Data Solutions, Inc., Tokyo (JP)

(72) Inventors: Tomoaki OHTSUKI, Yokohama-shi (JP); Kohei YAMAMOTO, Yokohama-shi (JP)

(73) Assignees: Data Solutions, Inc., Tokyo (JP); Data Solutions, Inc., Tokyo (JP)

(21) Appl. No.: 17/637,407

(22) PCT Filed: Aug. 20, 2020
(86) PCT No.: PCT/JP2020/031523

§ 371 Date: Feb. 22, 2022

(30) Foreign Application Priority Data
Aug. 23, 2019 (JP) 2019-152755

(51) Int. Cl. A61B 5/08; A61B 5/05; A61B 5/00

(52) U.S. Cl. CPC A61B 5/0816; A61B 5/05; A61B 5/7257; A61B 5/7264

(57) ABSTRACT

It is an object to improve the accuracy of detecting breathing. The present system for detecting breathing comprises: a transmission unit that transmits radio waves to a plurality of positions including a position at which a subject is present; a reception unit that receives reflected waves as a result of reflection of the radio waves; a phase fluctuation calculator that calculates phase fluctuation of the reflected waves; a generator that performs a Fourier transform on the phase fluctuation and generates a spectrogram indicating a relationship between a time at which the reflected waves are received and a frequency component included in the reflected waves; and a breathing rate estimator that estimates a breathing rate of the subject by outputting a probability that the subject takes breaths at a predetermined frequency for each frequency based on the spectrogram and calculating a weighted average of the frequency by using the probability as a weight.

### TECHNICAL FIELD

The present invention relates to a system for detecting breathing and a method for detecting breathing.

### BACKGROUND ART

In recent years, it has been known that technique that uses a radar or the like to detect biological information such as a breathing rate while the device is out of contact with a subject. The breathing rate or the like detected in this manner is used for monitoring health conditions, confirming survival, detecting disordered breathing or the like.

Specifically, a method for detecting breathing in a multiple-input multiple-output (MIMO) manner by using a frequency modulated continuous wave (FMCW) sensor has been known. There has been proposed a method for detecting breathing in such a manner even if a subject is not present in front of or on the line of sight (LOS) of a radar (see Non Patent Literature 1, for example).

### CITATION LIST

### Non Patent Literature

Non Patent Literature 1: A. Ahmad et al., “Vital signs monitoring of multiple people using a FMCW millimeter-wave sensor”, IEEE Int. Conf. on 2018 IEEE Radar Conference (RadarConf18), pp. 1450-1455, April 2018., [online], [retrieved on Jul. 30, 2019], Internet <URL: https://ieeexplore.ieee.org/abstract/document/8378778>

### SUMMARY OF INVENTION

### Technical Problem

An object of the present invention, which has been made in view of the above-described point, is to improve the accuracy of detecting breathing.

### Solution to Problem

The present system for detecting breathing is required to include:

a transmission unit that transmits radio waves to a plurality of positions including a position at which a subject is present;

a reception unit that receives reflected waves as a result of reflection of the radio waves;

a phase fluctuation calculator that calculates phase fluctuation of the reflected waves;

a generator that performs a Fourier transform on the phase fluctuation and generates a spectrogram indicating a relationship between a time at which the reflected waves are received and a frequency component included in the reflected waves; and

a breathing rate estimator that estimates a breathing rate of the subject by outputting a probability that the subject takes breaths at a predetermined frequency for each frequency based on the spectrogram and calculating a weighted average of the frequency by using the probability as a weight.

### Advantageous Effect of Invention

According to the disclosed technology, it is possible to improve the accuracy of detecting breathing.

### BRIEF DESCRIPTION OF DRAWINGS

FIG. 1 shows an example entire configuration of a system for detecting breathing.

FIG. 2 shows an example hardware configuration of an information processor in the system for detecting breathing.

FIG. 3 shows an example functional configuration of the system for detecting breathing.

FIG. 4 shows an example of off-line processing by the system for detecting breathing.

FIG. 5 shows an example of setting of a position targeted for detection by the system for detecting breathing.

FIG. 6 shows an example of transmitted radio waves and reflected waves.

FIG. 7 shows an example relationship between the transmitted radio waves and the reflected waves.

FIG. 8 shows an example of receiving the reflected waves.

FIG. 9 shows the calculation of phase fluctuation or the like.

FIG. 10A shows an example of a person-present spectrogram.

FIG. 10B shows an example of a person-present spectrogram.

FIG. 11A shows an example of a person-absent spectrogram.

FIG. 11B shows an example of a person-absent spectrogram.

FIG. 12 shows an example structure of a convolutional neural network in the system for detecting breathing.

FIG. 13 shows an example structure of a neural network in a comparative example.

FIG. 14 shows an example of on-line processing by the system for detecting breathing.

FIG. 15 shows an experiment environment.

FIG. 16 shows parameters for a MIMO FMCW radar in an experiment.

FIG. 17 shows experiment conditions.

FIG. 18 shows an experimental result.

FIG. 19 shows an example configuration in a learning phase.

FIG. 20 shows an example configuration in an execution phase.

### DESCRIPTION OF EMBODIMENT

The following describes an optimal and minimal embodiment for carrying out the invention with reference to the drawings. Note that, in the drawings, identical reference characters indicate similar components, and overlapping descriptions will be omitted. Specific examples shown in the drawings are illustrative, and any other components than those shown in the drawings may be included.

<Example Entire Configuration of System for Detecting Breathing>

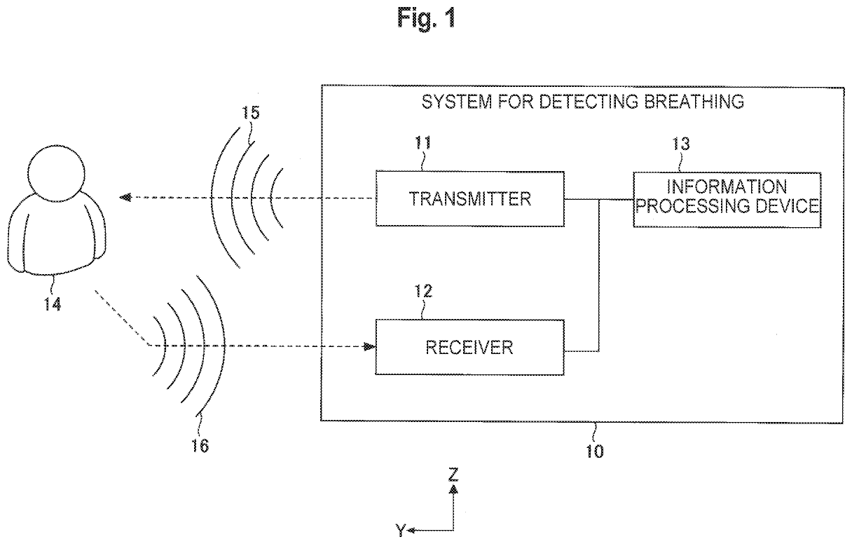

FIG. 1 shows an example entire configuration of a system for detecting breathing. For example, a system for detecting breathing 10 includes a transmitter 11, a receiver 12, and an information processor 13.

In the following description, the direction in which the transmitter 11 transmits radio waves 15 (the left-right direction in the figures) is defined as a “Y-axis direction”. The up-down direction, or the height direction, in the figures is defined as a “Z-axis direction”. The direction orthogonal to the Y-axis direction rightward (the depth direction in the figure) is defined as an “X-axis direction”.

The transmitter 11 transmits radio waves 15 toward a subject 14. For example, the transmitter 11 includes an antenna, an electronic circuit and the like.

The receiver 12 receives radio waves (hereinafter referred to as “reflected waves 16”) reflected from the subject 14 as a result of the impingement of the transmitted radio waves 15 on the subject 14. For example, the receiver 12 includes an antenna, an electronic circuit and the like.

The transmitter 11 and the receiver 12 as described above form a so-called MIMO FMCW radar or the like.

The information processor 13 performs signal processing on the reflected waves 16 received by the receiver 12. For example, the information processor 13 is a personal computer (PC), an electronic circuit or the like.

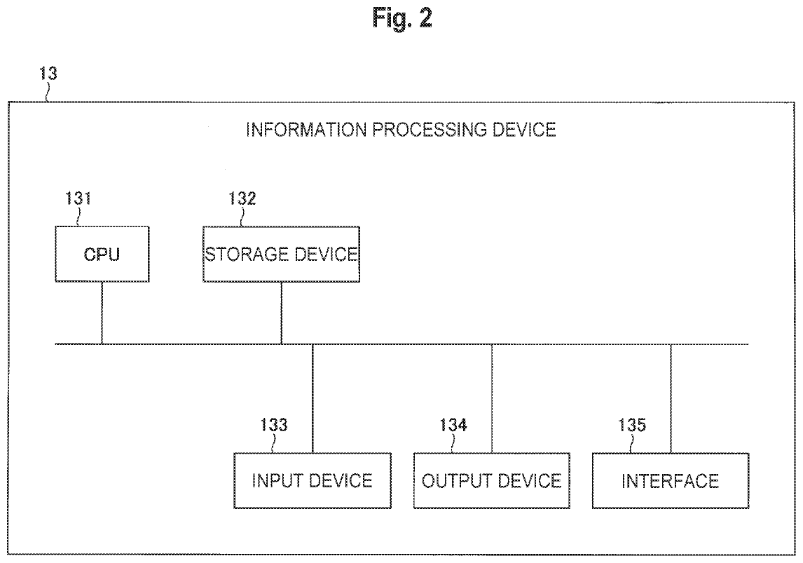

FIG. 2 shows an example hardware configuration of an information processor in the system for detecting breathing. For example, the information processor 13 has a hardware configuration including a central processing unit (CPU, hereinafter simply referred to as a “CPU 131”), a storage device 132, an input device 133, an output device 134, and an interface 135.

The CPU 131 is an example of a computing device and a control device.

The storage device 132 is a primary storage device such as a memory, for example. Note that the storage device 132 may further include an auxiliary storage device such as a hard disk.

The input device 133 is a device to which a user operation or the like is input. For example, the input device 133 is a keyboard, a mouse or the like.

The output device 134 is a device that outputs a processing result or the like to the user. For example, the output device 134 is a display or the like.

The interface 135 is a device that transmits and receives data to/from an external device via a cable, a network or the like through wired or wireless communication. For example, the interface 135 is a connector, antenna or the like.

Thus, the information processor 13 performs various processing or control operations by causing the computing device such as the CPU 131 and the storage device 132 or the like to cooperate based on a program or the like.

Note that the hardware configuration of the information processor 13 is not limited to the configuration shown in the figure. That is, the information processor 13 may have a hardware configuration further including, externally or internally, a computing device, a control device, a storage device, an input device, and an output device.

<Example Functional Configuration>

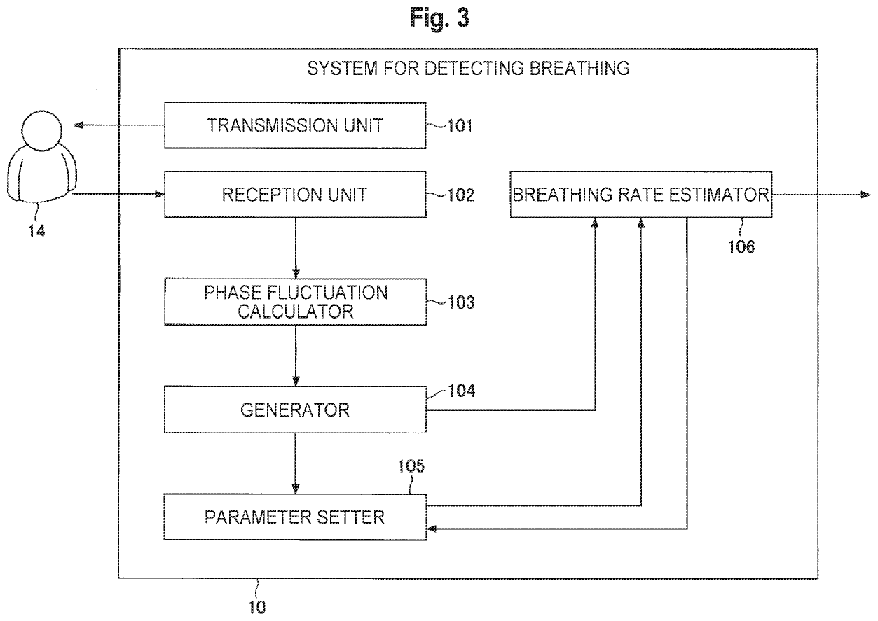

FIG. 3 shows an example functional configuration of the system for detecting breathing. For example, the system for detecting breathing 10 has a functional configuration including a transmission unit 101, a reception unit 102, a phase fluctuation calculator 103, a generator 104, and a breathing rate estimator 106. Note that, as shown in the figure, the system for detecting breathing 10 preferably has the functional configuration further including a parameter setter 105. The following description will be made with reference to the functional configuration shown in the figure as an example. The specific functions of the respective functional blocks will be described later.

The transmission unit 101 is implemented by the transmitter 11 or the like, for example.

The reception unit 102 is implemented by the receiver 12 or the like, for example.

The phase fluctuation calculator 103, the generator 104, the parameter setter 105, the breathing rate estimator 106 and the like are implemented by the information processor 13 or the like, for example.

Note that the system for detecting breathing 10 may further include any other functional components than those shown in the figure.

<Example of Off-Line Processing>

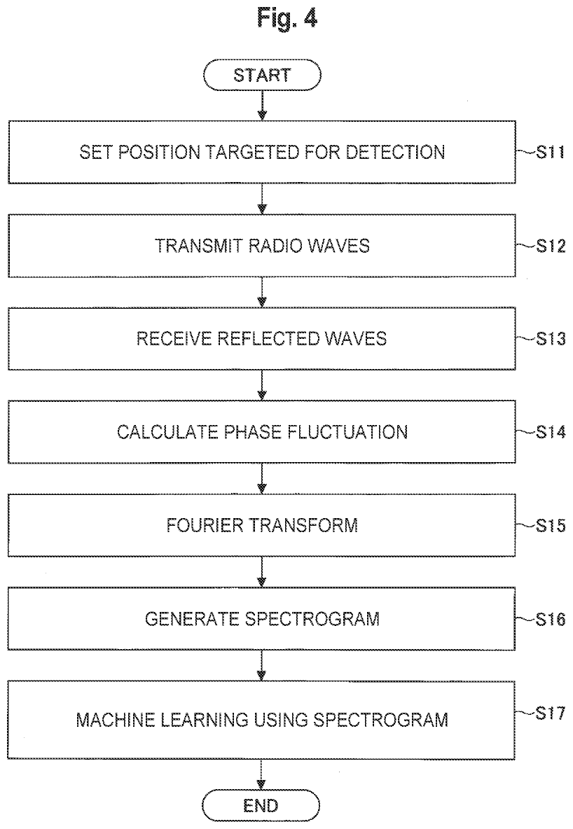

FIG. 4 shows an example of off-line processing by the system for detecting breathing. The off-line processing is performed as preprocessing prior to breathing rate estimation. That is, the off-line processing is performed as a preprocessing for processing performed on the precondition that the off-line processing has been performed in advance (so-called main processing or execution processing, hereinafter referred to as “on-line processing”). Also, the off-line processing is a processing including so-called learning processing.

(Example of Setting Position Targeted for Detection) (Step S11)

In step S11, a position targeted for detection, that is, the position of the chest wall (chest) of a person is set for the transmission unit 101 and the reception unit 102. For example, the position targeted for detection is set as described below.

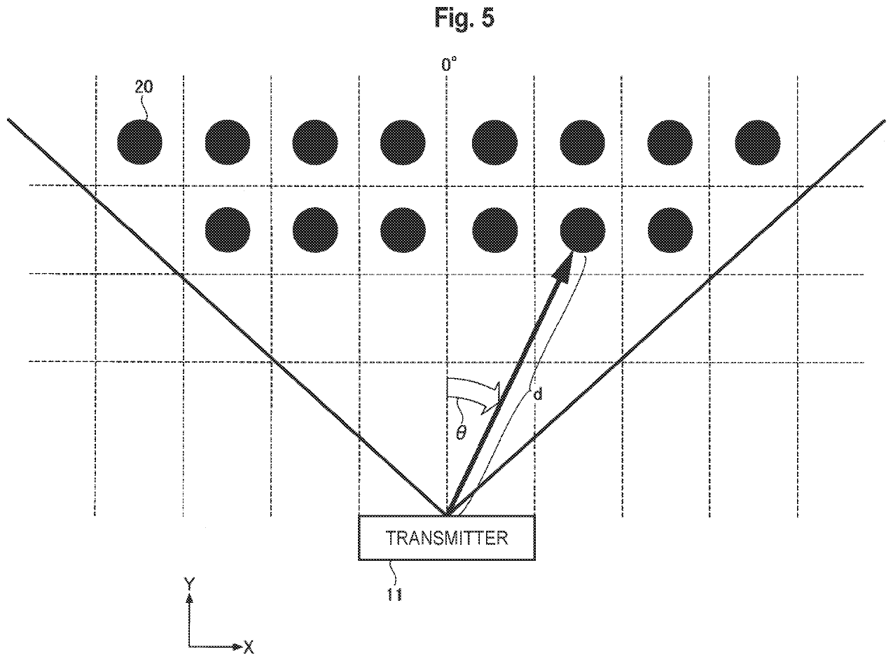

FIG. 5 shows an example of setting of a position targeted for detection by the system for detecting breathing. Hereinafter, a position 20 at the front of the transmitter 11 is defined as being at an angle θ of “0°”. In addition, in the figure, an angle on the right (i.e., in a clockwise direction) of “0°” is defined as a “plus” angle, while an angle on the left (i.e., in a counterclockwise direction) of “0°” is defined as a “minus” angle.

In addition, the distance from the transmitter 11 to the position 20, that is, the distance traveled by radio waves is defined as a “distance d”. Therefore, assuming that the height (the position in the Z-axis direction) is fixed, each position 20 is uniquely determined on a plane (i.e., on an X-Y plane) if the distance d and the angle θ are determined.

For example, by setting positions 20 to which radio waves are transmitted at every predetermined distance and at every predetermined angle, each position 20 targeted for detection and a detection range can be set. Note that the method for setting the position may not be the method for setting the distance d and the angle θ. For example, the position 20 may be set by inputting a coordinate value or the like.

In addition, it is preferable that the angle θ is set at every 10° and the distance d is set at every 0.1 m. It is preferable that each divided space corresponds to about one person. Therefore, an appropriate space for the size of a person can often be set if radio waves are emitted at every 10° and at every 0.1 m.

(Example of Transmitting Radio Waves) (Step S12)

In step S12, the transmission unit 101 transmits radio waves. That is, the transmission unit 101 transmits radio waves to each position 20 determined in step S11.

Note that the position targeted for detection or the like is determined as shown in FIG. 5 in step S11 or the like, for example. However, for example, at the time that a sensor is disposed, the range targeted for detection may be roughly determined. The position determination as in FIG. 5 may then be performed in step S12 or in a subsequent process or the like, for example.

(Example of Receiving Reflected Waves) (Step S13)

In step S13, the reception unit 102 receives reflected waves. That is, the reception unit 102 receives reflected waves of the radio waves transmitted in step S12.

(Example of Calculating Phase Fluctuation) (Step S14)

In step S14, the phase fluctuation calculator 103 calculates the phase of the reflected waves fluctuating with the movement of the chest wall due to breathing, that is, phase fluctuation.

(Example of Fourier Transform) (Step S15)

In step S15, the generator 104 performs a Fourier transform on the phase fluctuation. For example, the generator 104 performs a transform such as a short time Fourier transform (STFT).

(Example of Generating Spectrogram) (Step S16)

In step S16, the generator 104 generates a spectrogram.

Steps S12 to S16 are performed as described below, for example.

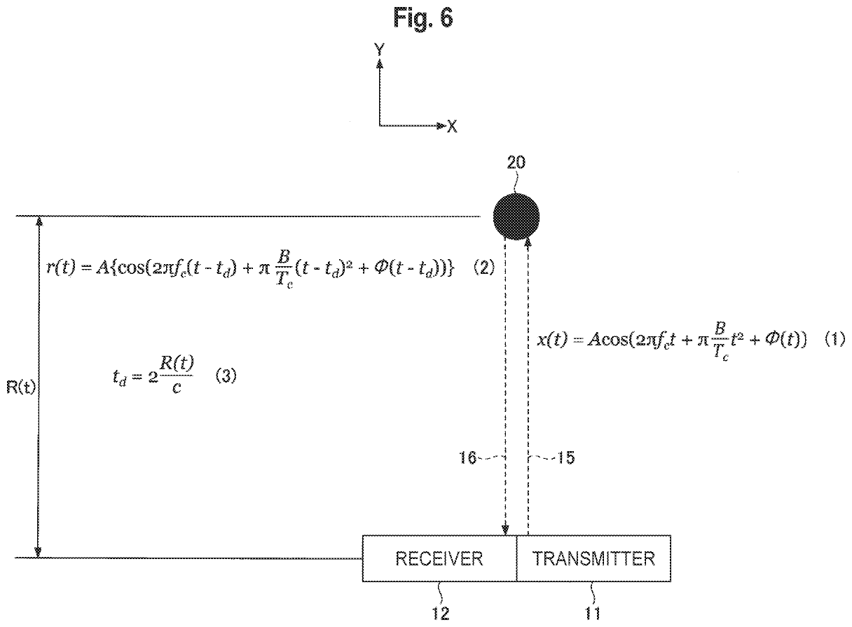

FIG. 6 shows an example of transmitted radio waves and reflected waves. For example, the radio waves 15 transmitted from the transmitter 11 to the position 20 in step S12 are in the form of a signal such as “x(t)” shown in equation (1). On the other hand, the reflected waves 16 are in the form of a signal such as “r(t)” shown in equation (2). Hereinafter, time is defined as “t”. The distance between any point of the position 20 targeted for detection and the transmitter 11 and the receiver 12 (although they are shown as being separate in the figure, the description will be made with reference to an example in which the transmitter 11 and the receiver 12 are at the same position, that is, the radio waves are transmitted and received at the same position) is defined as “R(t)”.

In equations (1) and (2), “A” indicates the strength of the received signal. In addition, the values of “B”, “Tc”, “fc”, “td”, and “fb” in equation (1) and the like are as described below.

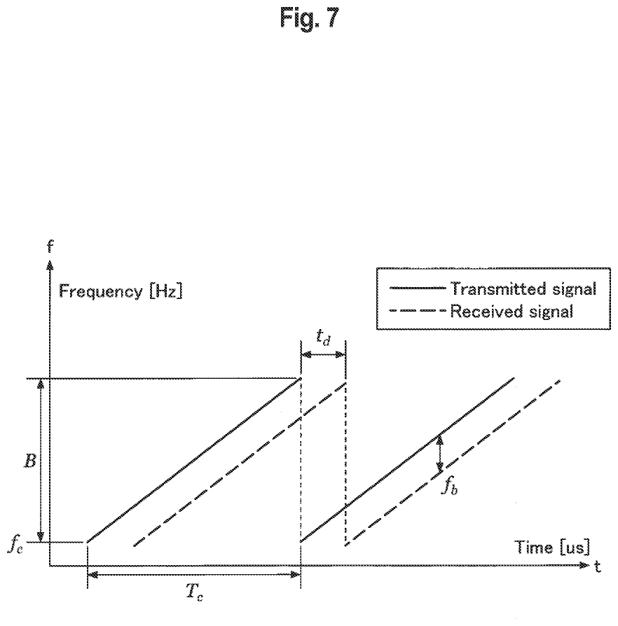

FIG. 7 shows an example relationship between the transmitted radio waves and the reflected waves. For example, the radio waves 15, that is, “x(t)” is set to have a value that changes with time in a range of frequency change width “B” (so-called sweep).

The “fc” is a so-called initial frequency. Therefore, the frequency of the radio waves 15 changes with time “t” in a range of frequency change width “B” relative to the initial frequency “fc”.

The “Tc” is a so-called sweep time. The sweep time “Tc” refers to the time during which the frequency of the radio waves 15 changes with time “t” from the initial frequency “fc” and returns to “fc”.

The “td” is a value as shown in equation (3). Hereinafter, “td” is referred to as a “reception time”. That is, the reception time “td” is the time from when the radio waves 15 are transmitted from the transmitter 11 to when the reflected waves 16 are received at the receiver 12.

The “c” indicates the speed of electromagnetic waves. Hereinafter, the “c” is referred to as an “electromagnetic wave speed”.

The reflected waves 16 are received as described below.

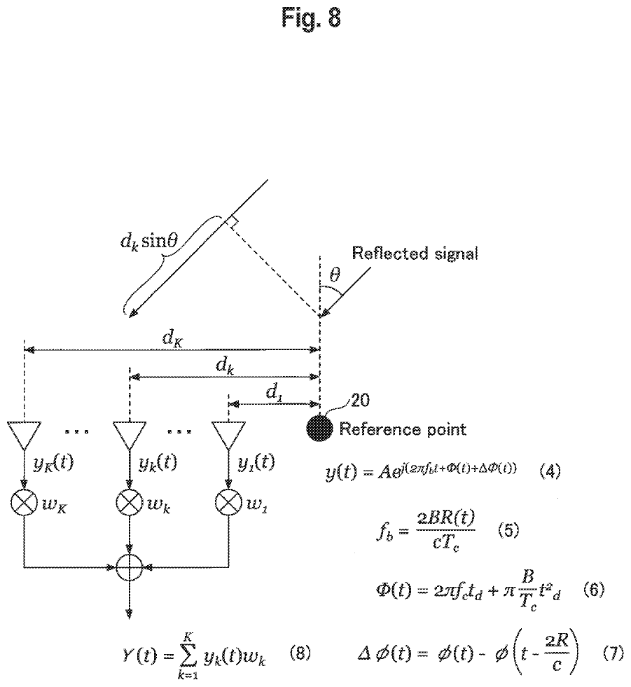

FIG. 8 shows an example of receiving the reflected waves. For example, the receiver receives the reflected waves at a plurality of distances between the receiver and the position 20 such as “d₁ . . . dₖ . . . dₓ”. Defining the frequency of the reflected waves received at each position as “y₁(t)” . . . “yₖ(t)” . . . “yₓ(t)”, each reflected wave frequency “y(t)” can be represented as in equation (4).

The “fb” in equation (4) or the like is the difference in frequency between the transmitted radio waves and the reflected waves (hereinafter simply referred to as a frequency difference “fb”). For example, the frequency difference “fb” is a value as shown in equation (5).

The “Φ(t)” in equation (4) or the like indicates the phase. For example, the phase “Φ(t)” is a value as shown in equation (6).

The “Δφ(t)” in equation (4) or the like indicates the phase fluctuation. For example, the phase fluctuation “Δφ(t)” is a value as shown in equation (7).

The weight coefficient for the “k”-th receiver of the respective receivers is defined as “wk”. The weight coefficient “wk” is a preset value. By using such a weight coefficient “wk”, reflected waves “Y(t)” for a certain angle can be calculated by a weighted average as shown in equation (8), for example.

The phase fluctuation calculator calculates the respective phase fluctuation as described below, for example, based on the results of receiving the reflected waves at various positions, that is, “y₁(t)” . . . “yₖ(t)” . . . . “y_{K}(t)”.

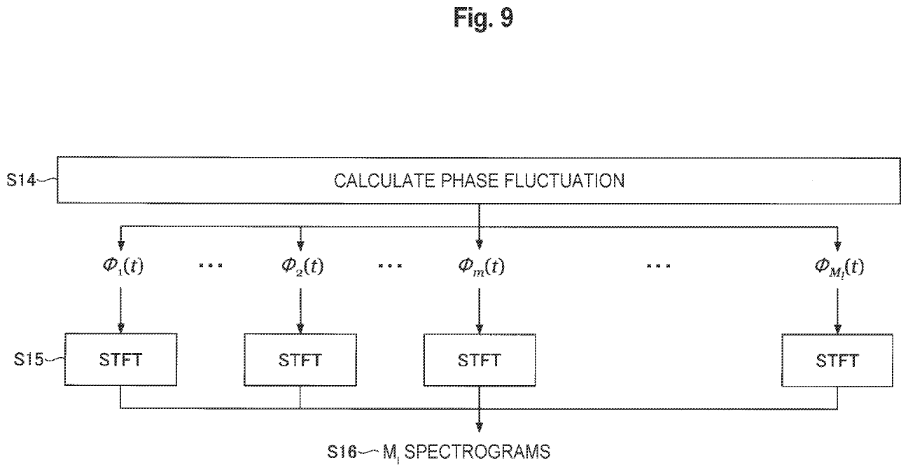

FIG. 9 shows the calculation of phase fluctuation or the like. For example, if reception is performed at “M₁” positions, phase fluctuation “φ₁(t)” . . . “φₘ(t)” . . . “φ_{M1}(t)” is calculated from the respective reception results (step S14).

Next, an SIFT is performed on the phase fluctuation “φ₁(t)” . . . “φₘ(t)” . . . “φ_{M1}(t)” (step S15), and “M1” spectrograms can then be generated.

Note that each of “Φ(t)”, “φₘ(t)” and “φ(t)” indicates the phase. However, “Δφ(t)” (indicated by a variable without a subscript) or the like indicates a part of the phase fluctuation “φₘ(t)” (indicated by a variable with a subscript) decomposed by elements.

Note that it is preferable that the spectrograms are generated separately for the case in which a person is present at the position targeted for detection and the case in which not present, as described below, for example.

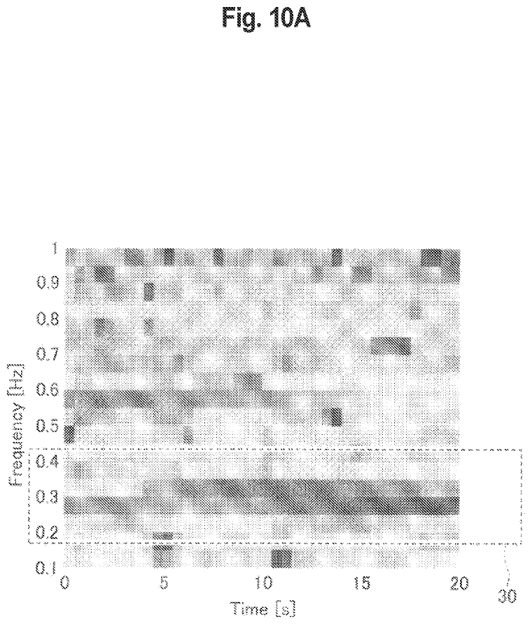

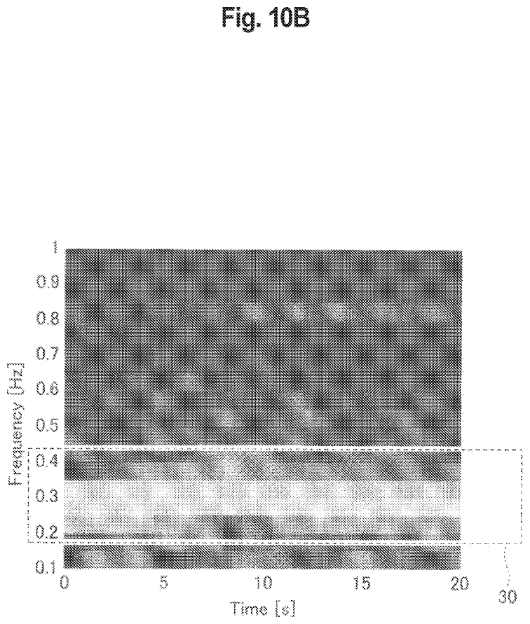

FIGS. 10A and 10B show examples of person-present spectrograms. In the figures, the vertical axis indicates the frequency, and the horizontal axis indicates the time of reception. For example, if a person is present at a position set in step S11, a spectrogram such as that in FIG. 10A or 10B is often generated. Hereinafter, such a spectrogram generated in the presence of a person is referred to as a “person-present spectrogram”. On the other hand, a spectrogram generated in the absence of a person is referred to as a “person-absent spectrogram”.

A spectrogram indicates the strength for each frequency included in the reflected waves with respect to time. Therefore, a spectrogram indicates what frequency components are included (distribution of strength on the vertical axis) in the received reflected waves at the same time (at the same value on the horizontal axis) with respect to time.

As indicated on the horizontal axis, the spectrogram is preferably generated per unit of time of 15 to 50 seconds such as 20 seconds. The unit of the horizontal axis in the figure, that is, the time at which the spectrogram is generated is the window size of the STFT in step S15. Therefore, the spectrograms shown in FIGS. 10A and 10B are both examples of spectrograms generated by an STFT with a window size set to 20 seconds.

Note that the spectrograms shown in FIGS. 10A and 10B are examples in which the step size is 0.5 seconds.

For example, if a Fourier transform can be performed by an STFT with a window size of about 20 seconds, the frequency of breathing can be detected in units of about 0.1 Hz. The breathing rate of a person is, in most cases, in a range of 0.1 Hz (meaning that the subject takes six breaths in one minute and the subject is in a resting state or the like) to 1.0 Hz (meaning that the subject takes 60 breaths in one minute and the subject has taken exercise or the like).

In addition, if the window size exceeds 50 seconds, that is, about one minute or more, the breathing rate of the subject often changes during the generation of the spectrogram.

Therefore, if the window size is set to a value greater than 50 seconds, the breathing rate of the subject often changes before processing. On the other hand, if the window size is about 15 to 50 seconds, the breathing rate can be accurately detected. That is, the detection can be performed before the breathing rate of the subject significantly changes, and a resolution suitable for detecting the breathing rate can be obtained.

The person-present spectrogram often assumes high strength at about 0.2 Hz to 0.4 Hz around about 0.3 Hz. However, frequencies at which high strength appears depend on the health conditions or motion conditions of a person, for example. Hereinafter, a range in which high strength appears is referred to as a “high-strength band 30”. That is, in the person-present spectrogram, the high-strength band 30 is often a range of 0.3 Hz and multiples of 0.3 Hz as shown in the figure, for example.

The high-strength band 30 includes frequencies at which the chest wall moves due to breathing of the person. That is, this example is generated in the case where a person taking breaths at 0.3 Hz is present at the detection position.

On the other hand, in the case where a person is absent, a person-absent spectrogram as described below is generated.

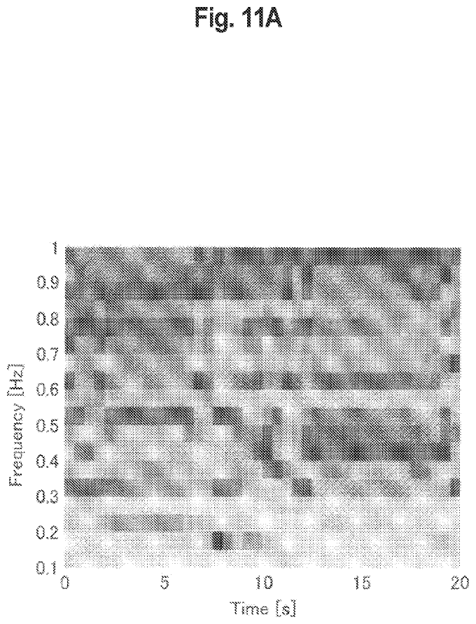

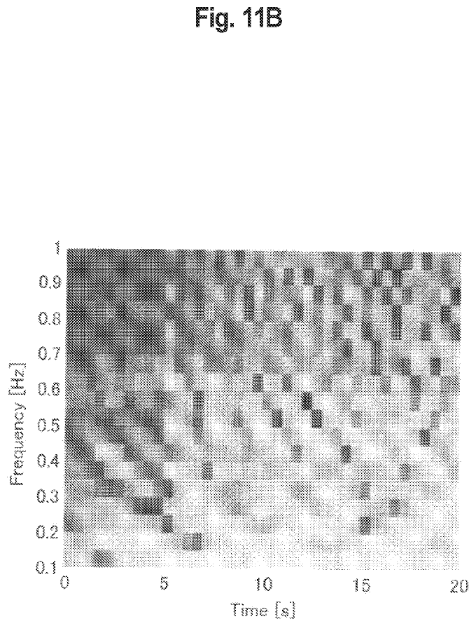

FIGS. 11A and 11B show examples of person-absent spectrograms. The person-absent spectrograms are different from the person-present spectrograms in that those shown in FIGS. 11A and 11B both have no high-strength band.

It is preferable to perform machine learning by using both of such person-present spectrograms and person-absent spectrograms. Thus, by performing machine learning for both of the person-present spectrograms and the person-absent spectrograms, it is possible to accurately learn the environment in which the detection is performed, that is, effects of furniture in a room in which the detection is performed or the like. That is, by using both of the person-present spectrograms and the person-absent spectrograms as so-called training data, it is possible to learn the environmental effects by machine learning, and thus to more accurately detect the breathing rate.

(Example of Machine Learning Using Spectrograms) (Step S17)

In step S17, the parameter setter 105 performs machine learning by using spectrograms.

Note that it is preferable that processing in the parameter setter 105 is performed in a convolutional neural network (CNN) as described below.

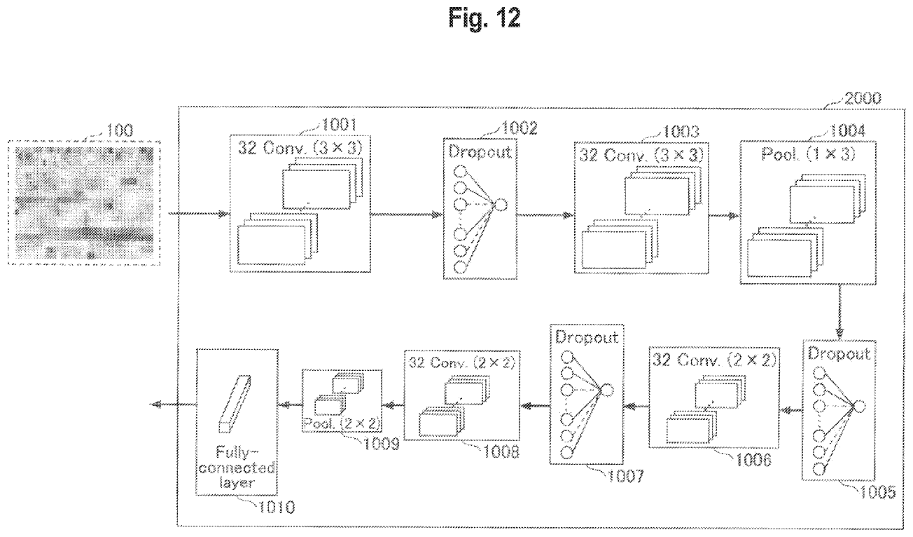

FIG. 12 shows an example structure of a convolutional neural network in the system for detecting breathing. As described above, it is preferable that the parameter setter 105 performs learning by using a neural network suitable for image processing.

A spectrogram 100 is input to a convolutional neural network 2000. It is preferable that, if the spectrogram 100 is input, the parameter setter 105 then performs learning in a processing order and a processing configuration as described below in learning processing.

It is preferable that, in the convolutional neural network 2000, processing is performed in the order of convolution 1001, dropout 1002, convolution 1003, pooling 1004, dropout 1005, convolution 1006, dropout 1007, convolution 1008, and pooling 1009, and then the processing of full connection 1010 is performed. However, in the convolutional neural network 2000, further processing other than shown in the figure may be performed.

The convolution is, for example, processing in which pixels of a target image (in this example, the spectrogram 100 is treated as an image. Hereinafter, an example in which the spectrogram 100 is treated as an image will be described. Therefore, in this example, data indicating the strength of the spectrogram 100 at each frequency with respect to time is treated as “pixels”) are multiplied by filter coefficients by using a preset filter (note that the filter coefficients set to the filter may be updated by learning). That is, in the convolution, processing called sliding window or the like is performed by using a filter. If the convolution processing is performed in this manner, a so-called feature map is output.

For example, in the convolution 1001 and the convolution 1003, “32 Conv. (3×3)” indicates setting to use “32” filters with a size of “3×3”. On the other hand, the convolution 1006 and the convolution 1008 are different in that they are “32 Conv. (2×2)” and the filter size is “2×2”.

The dropout is processing for preventing so-called overtraining. Overtraining is a phenomenon in which optimization is performed to features included only in training data and accuracy for actual data is decreased. For example, the dropout is processing of disconnecting some of the connections between the full connection and the output layer, or the like.

Specifically, the dropout 1002 prevents some of the outputs of the convolution 1001 from being input to the convolution 1003 or the like in the subsequent processing, or the like. Note that what outputs of the outputs of the convolution 1001 are to be prevented by the dropout 1002 from being output to the subsequent process is randomly selected, for example. Similarly, the dropout 1005 and the dropout 1007 also prevent some of the outputs of the pooling 1004 or the convolution 1006 in the preceding processing from being input to the subsequent processing. Note that what proportion is subjected to the dropout, that is, a parameter such as a dropout rate is preset to “25%”, for example.

The pooling is maximum pooling (max pooling) or the like, for example. In the “max pooling”, a pixel with the maximum value of the pixels in a predetermined window is extracted. By performing pooling on the image subjected to convolution in this manner, data of the image can be compressed. That is, the pooling serves as so-called downsizing or the like.

Further, by performing the pooling, small differences in a predetermined window are often absorbed, and thus it is possible to robustly recognize features even if there are small position changes of about several pixels. In addition, by performing the pooling, the quantity of data is often decreased, and thus it is possible to reduce the computation cost in the subsequent processing or the like.

For example, in the pooling 1004, “Pool. (1×3)” indicates that a window with a size of “1×3” is used. Similarly, in the pooling 1009, “Pool. (2×2)” indicates that a window with a size of “2×2” is used.

The full connection performs processing such as weighting a plurality of outputs from the preceding processing and calculating a sum. In the full connection, weights and the like are set by an activation function or the like. The activation function is a rectified linear unit (ReLU, ramp function) or the like, for example.

In the full connection 1010, the output results from the preceding pooling 1009 are calculated by the activation function or the like. For example, the full connection 1010 outputs a probability for each of 11 frequency types of “0.1 Hz”, “0.2 Hz”, . . . , “0.9 Hz”, “1.0 Hz”, and “no breathing (synonymously a frequency of “0 Hz”)”. That is, in the convolutional neural network 2000, a probability that the subject takes breaths at each frequency is calculated. Then, by performing calculation of weighting each probability, that is, calculating a weighted average, the breathing rate of the subject at the detection position can be detected.

It is preferable that a frequency region to determine that breathing is performed, that is, the high-strength band 30 or the like is learned, optimized as a result of the learning, and set as a parameter, so that the breathing rate can be accurately detected by machine learning. Note that the parameter may be related to a dropout rate or the like.

Note that it is preferable that parameters to be changed based on machine learning include a filter size used for convolution and pooling. The filter size is a parameter that significantly effects the accuracy. Therefore, the accuracy can be improved by optimizing the filter size.

Note that the breathing rate is not necessarily detected in the form of frequency. For example, the breathing rate may be output in a form such as “a breathing rate per unit time (e.g., per minute or the like)” (in the unit of “times/minute” or the like) or a value obtained by averaging some estimation results (which may also be a statistical value such as a moving average).

### Comparative Example

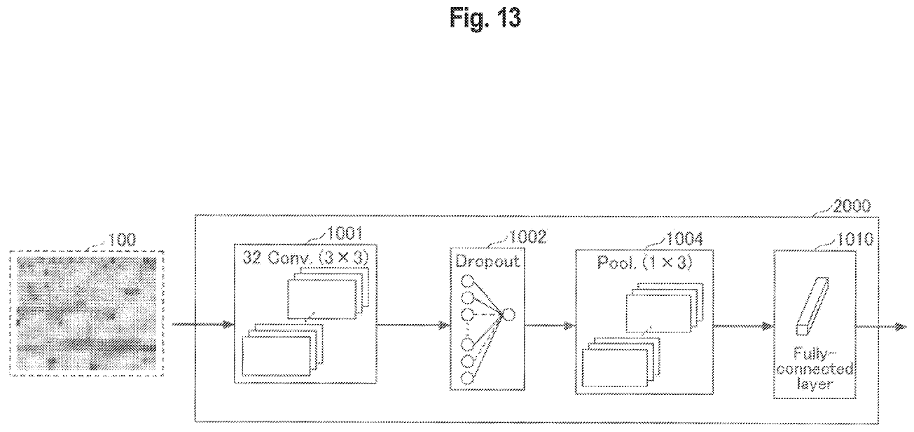

FIG. 13 shows an example structure of a neural network in a comparative example. For example, the comparative example will be described with reference to a structure including about one process for each of convolution, dropout, pooling, and full connection. The comparative example is different from FIG. 12 in the order of performing processes such as convolution, dropout, and pooling, and the number of processes and the like.

With a neural network with a structure as in the comparative example, it is often impossible to obtain sufficient accuracy.

<Example of On-Line Processing>

On-line processing is processing performed after the off-line processing is performed, that is, after learning is performed by the learning processing. That is, the on-line processing is main processing using actual data in contrast to the off-line processing, which is learning processing using training data.

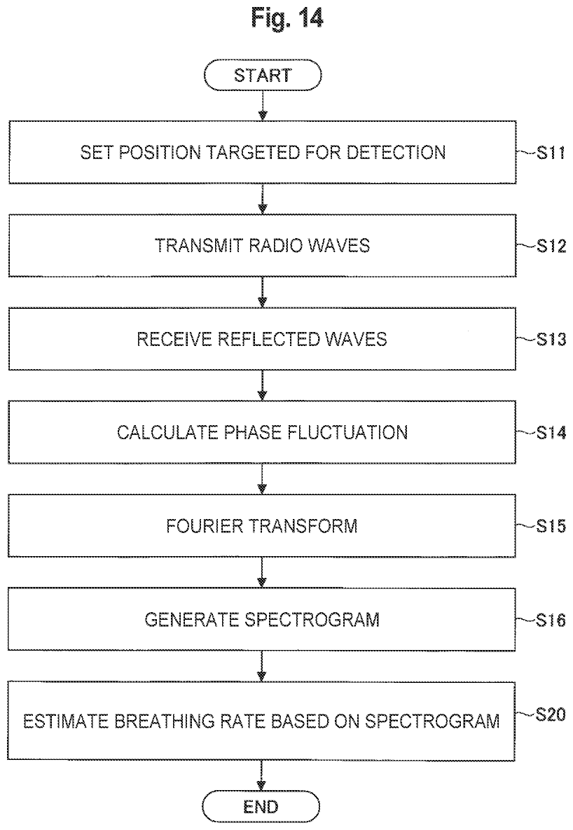

FIG. 14 shows an example of on-line processing by the system for detecting breathing. It is different from the off-line processing in that it is performed in step S20. In the following, differences will be mainly described, and the description of processing similar to the off-line processing will be omitted.

(Example of Estimating Breathing Rate Based on Spectrogram) (Step S20)

In step S20, the breathing rate estimator 106 estimates the breathing rate based on a spectrogram generated by using actual data. A result of an experiment of estimating the breathing rate by such processing is shown below.

<Experimental Result>

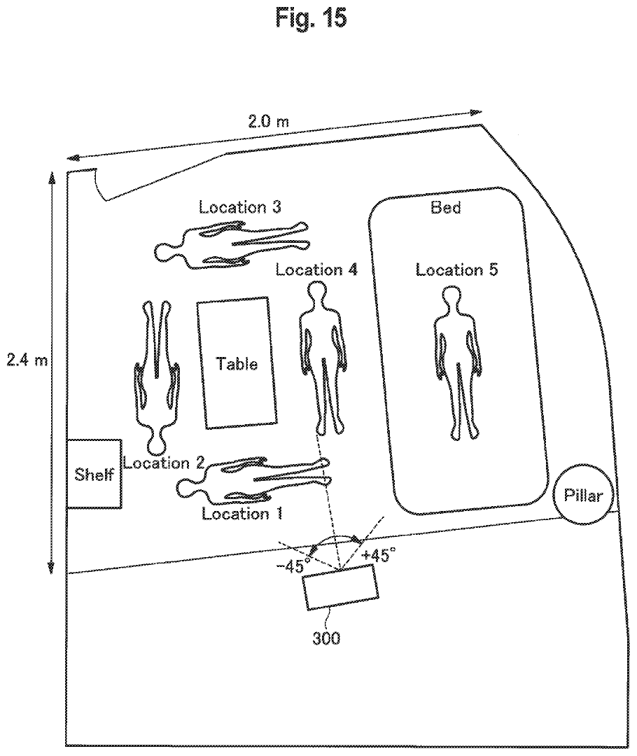

FIG. 15 shows an experiment environment. In this experiment, a MIMO FMCW radar 300 as an example of the transmitter and the receiver is used. The MIMO FMCW radar 300 is a device having parameters described below.

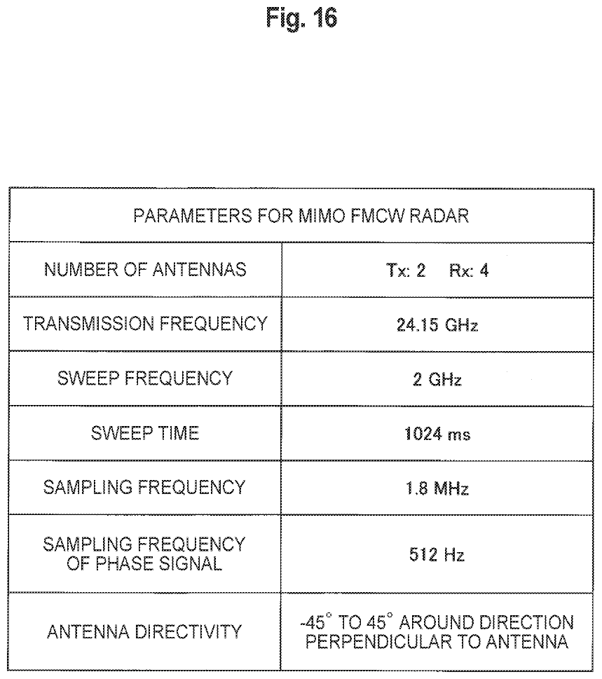

FIG. 16 shows parameters for the MIMO FMCW radar in the experiment.

The “Tx” of “NUMBER OF ANTENNAS” indicates the number of antennas for transmission. Thus, the radio waves were transmitted by “two” antennas in this experiment.

The “Rx” of “NUMBER OF ANTENNAS” indicates the number of antennas for reception. Thus, the reflected waves were received by “four” antennas in this experiment.

The “TRANSMISSION FREQUENCY” indicates the frequency of the transmitted radio waves. Note that the frequency used for the radio waves may be a frequency other than “24.15 Hz”. For example, the frequency may be about 100 MHz to 100 GHz.

The “SWEEP FREQUENCY” indicates the width of swept frequencies. It corresponds to “B” in FIG. 7.

The “SWEEP TIME” indicates the time for which the sweep is performed. It corresponds to “Tc” in FIG. 7.

The “SAMPLING FREQUENCY” indicates the resolution of sampling the reflected waves.

The “SAMPLING FREQUENCY OF PHASE SIGNAL” indicates the frequency of sampling phase fluctuation signals calculated in step S14.

The “ANTENNA DIRECTIVITY” indicates the angle at which the radio waves are transmitted and received.

The experiment was performed under experiment conditions described below by using the positions of “Location 1” to “Location 5” in the experiment environment shown in FIG. 15 as detection positions.

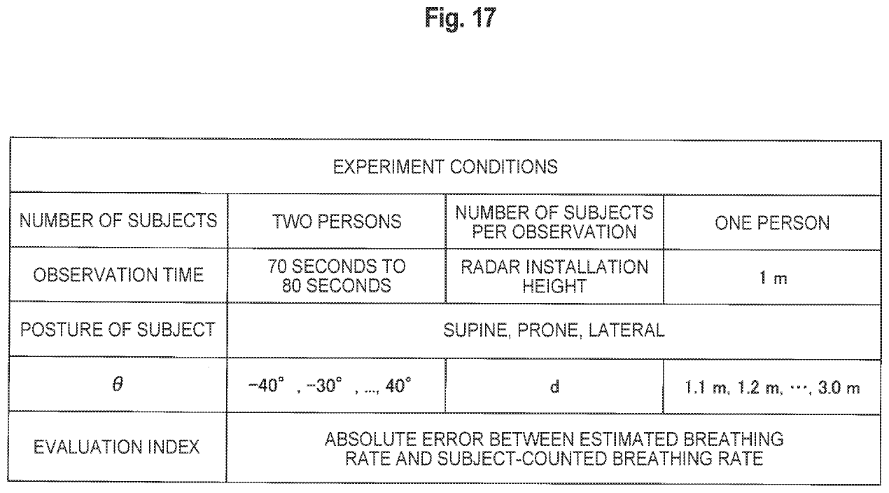

FIG. 17 shows experiment conditions.

As indicated by “NUMBER OF SUBJECTS”, this experiment was mainly performed for the case where two persons are present at the positions “Location 1” to “Location 5”.

The “NUMBER OF SUBJECTS PER OBSERVATION” indicates the number of subjects that are simultaneously present at each position. This experiment was performed in the case of “ONE PERSON”, that is, in the case where one subject is present at each position.

The “observation time” indicates the time for which data is measured, that is, the time for which the radio waves are transmitted and the reflected waves are received.

The “RADAR INSTALLATION HEIGHT” indicates the height at which the MIMO FMCW radar 300 is installed. That is, it indicates that in this experiment, the radio waves and the reflected waves were transmitted and received at a height of “1 m”.

The “POSTURE OF SUBJECT” indicates that three types of posture “supine”, “prone”, and “lateral” were taken.

The “θ” indicates the angle at which the radio waves are transmitted. As shown in the figure, the radio waves were transmitted at every 10° in this experiment.

The “d” indicates the distance traveled by the radio waves. As shown in the figure, the radio waves were transmitted at every 0.1 m in this experiment.

The “evaluation index” indicates an index for evaluating the breathing rate estimated by the system for detecting breathing. As shown in the figure, the subject counts the number of breaths he/she took, and a breathing rate reported by the subject is assumed as a “correct” breathing rate. Therefore, the breathing rate reported by the subject and the breathing rate estimated by the system for detecting breathing are compared, and their difference is calculated as an absolute error. The less the value of the absolute error is, the higher the accuracy is evaluated to be.

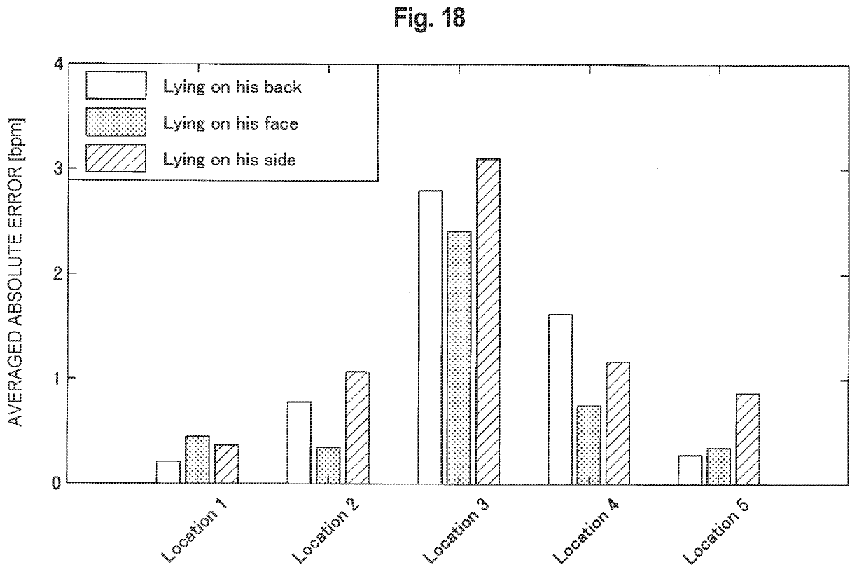

FIG. 18 shows an experimental result. The horizontal axis indicates the position of the subject by “Location 1” to “Location 5”. On the other hand, the vertical axis indicates the absolute error, that is, the evaluation result. Note that the unit of the vertical axis is “breath per minute”, that is, “times/minute”.

As shown in the figure, the system for detecting breathing can accurately estimate the breathing rate. Specifically, for “Location 1”, the averaged absolute error was “0.34 bpm”. For “Location 2”, the averaged absolute error was “0.72 bpm”. For “Location 4”, the averaged absolute error was “1.18 bpm”. For “Location 5”, the averaged absolute error was “0.50 bpm”.

Thus, even in an indoor environment or the like in which furniture and the like such as a “Bed”, a “Table”, a “Shelf”, and a “Pillar” are arranged, it is possible to improve the accuracy of detecting breathing.

<Example of Including Mechanism>

The system for detecting breathing may further include a mechanism for changing the angle of the radio waves transmitted by the transmission unit. Specifically, the system for detecting breathing includes an actuator for an antenna or the like for transmitting radio waves, a mechanism component or the like for changing the orientation of the antenna, or the like. That is, the mechanism may be configured to automatically change the angle by an actuator or the like or may be configured to be capable of manually changing the orientation of the antenna. Such a mechanism allows the system for detecting breathing to change the angle of transmitting the radio wave or the like. Therefore, it is possible to transmit the radio waves toward positions at different angles even with one antenna.

Thus, the configuration including the mechanism allows the system for detecting breathing to have a reduced number of components such as an antenna for realizing the transmission unit.

<Difference in Configuration Between Execution Phase and Learning Phase>

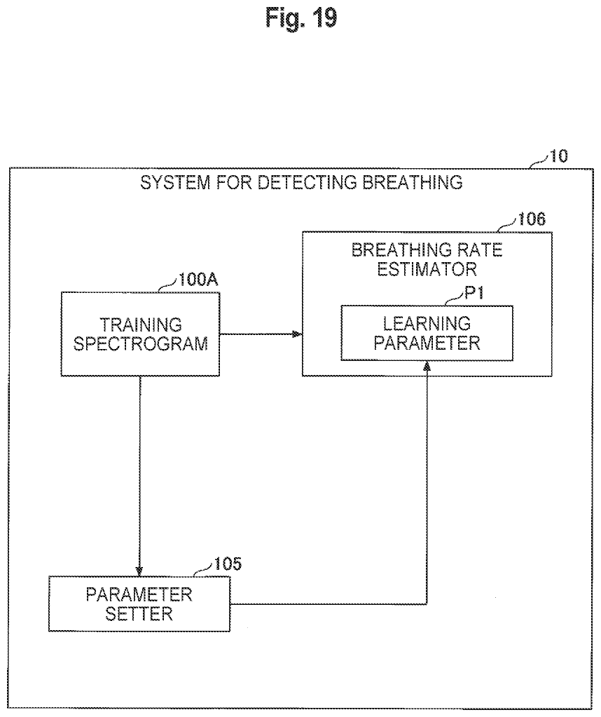

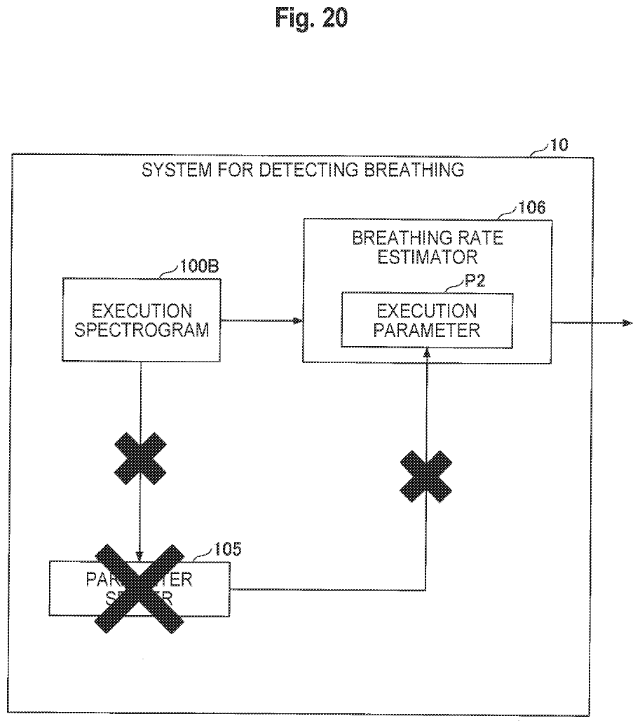

As shown in FIGS. 19 and 20, the system for detecting breathing may have a different configuration for each of the case of performing machine learning and the case of execution with parameters set by the machine learning or the like, that is, in a so-called trained state.

FIG. 19 shows an example configuration in a learning phase. For example, the configuration for performing the processing as shown in FIG. 4, that is, the configuration for performing machine learning is achieved by the configuration as shown in the figure.

For example, the parameter setter 105 includes a so-called learning unit or the like, and performs learning by using a training spectrogram 100A as training data.

For example, a parameter used by the breathing rate estimator 106 is updated based on a result of the machine learning using the training spectrogram 100A. That is, the parameter setter 105 sets a parameter for the breathing rate estimator 106 based on the training spectrogram 100A. Hereinafter, a parameter in the learning phase is referred to as a “learning parameter P1”. Thus, in machine learning such as CNN, the input spectrogram or the like is compressed by the convolution layer, the pooling layer or the like, and the compressed data is associated with a true label. In the learning phase, such processing is repeated for each piece of training data to optimize the parameter or the like.

A parameter used in the execution phase (hereinafter referred to as an “execution parameter P2”) is generated by performing a plurality of updates to the learning parameter P1 or the like by machine learning, for example.

However, the execution parameter P2 is not necessarily limited to that generated only by machine learning. For example, the execution parameter P2 may be generated by further manually modifying a parameter generated by the learning phase.

The execution parameter P2 may also be updated by performing machine learning by using an execution spectrogram 100B. That is, the execution parameter P2 is first generated by a machine learning method or a method other than machine learning and input externally. Thereafter, it may also be updated by performing machine learning by using an execution spectrogram 100B. In this case, evaluation may be input for an output even in the execution phase.

FIG. 20 shows an example configuration in the execution phase. The execution phase is different from the learning phase in that the parameter setter 105 is not connected. For example, if the parameter setter 105 is implemented by a program, the parameter setter 105 may be configured not to update the execution parameter P2 in the execution phase, for example. However, if it is configured to update the execution parameter P2, the execution phase may be performed with a program for realizing the parameter setter 105 installed.

### Other Embodiments

For example, the transmitter, the receiver, or the information processor may be a plurality of devices. That is, processing and control operations may be virtualized, parallelized, distributed or redundantized. On the other hand, the transmitter, the receiver and the information processor may have integrated hardware or share a device.

The parameter setter may change the breathing estimation unit, that is, the trained model or the like as a parameter by learning. Specifically, the parameter setter may change a parameter for the breathing estimation unit based on a learning result. Such a configuration in which learning results are applied to the breathing estimation unit may be used.

For the update by learning in the breathing estimation unit, a computing device included in a device constituting the system for detecting breathing may perform processing and control. On other hand, the update by learning in the breathing estimation unit may be performed by a learning unit or the like connecting via a network or the like. That is, the learning unit or the like may be implemented by cloud computing or the like that can be used via a network or the like.

Further, the parameter setter may perform the learning processing or the like by using a configuration other than a neural network by machine learning or the like. Specifically, the system for detecting breathing may use so-called artificial intelligence (AI) or the like. For example, the parameter setter may be implemented by a structure that performs machine learning such as a generative adversarial network (GAN), a recurrent neural network (RNN), or long short-term memory (LSTM). A machine learning algorithm such as random forests may also be used.

Note that all or part of each process according to the present invention may be written in a low-level language such as an assembler or a high-level language such as an object-oriented language and implemented by a program for causing a computer to perform a method for detecting breathing. That is, the program is a computer program for causing a computer of the system for detecting breathing or the like including a breathing detection device, the information processor or the like to perform each process.

Therefore, if each process is performed based on the program, a computing device and a control device included in the computer perform computation and control based on the program in order to perform each process. A storage device included in the computer stores data used for the process based on the program in order to perform each process.

The program can be recorded on a computer-readable recording medium and distributed. Note that the recording medium is a medium such as a magnetic tape, a flash memory, an optical disk, a magneto-optical disk or a magnetic disk. Further, the program can be distributed through telecommunication lines.

Although a preferred embodiment or the like has been described in detail above, there is no limitation to the above-described embodiment or the like, and various modifications and replacements can be made to the above-described embodiment or the like without departing from the scope of the claims.

This international application claims priority based on Japanese Patent Application No. 2019-152755 filed on Aug. 23, 2019, which is hereby incorporated herein by reference.

### REFERENCE SIGNS LIST

10 system for detecting breathing
11 transmitter
12 receiver
13 information processor
14 subject
15 radio waves
16 reflected waves
20 position
30 high-strength band
100 spectrogram
101 transmission unit
102 reception unit
103 phase fluctuation calculator
104 generator
105 parameter setter
106 breathing rate estimator
300 FMCW radar
1001 convolution
1002 dropout
1003 convolution
1004 pooling
1005 dropout
1006 convolution
1007 dropout
1008 convolution
1009 pooling
1010 full connection
2000 convolutional neural network
d distance
θ angle

1. A system for detecting breathing comprising:
a transmission unit that transmits radio waves to a plurality of positions including a position at which a subject is present;
a reception unit that receives reflected waves as a result of reflection of the radio waves;
a phase fluctuation calculator that calculates phase fluctuation of the reflected waves;
a generator that performs a Fourier transform on the phase fluctuation and generates a spectrogram indicating a relationship between a time at which the reflected waves are received and a frequency component included in the reflected waves; and
a breathing rate estimator that estimates a breathing rate of the subject by outputting a probability that the subject takes breaths at a predetermined frequency for each frequency based on the spectrogram and calculating a weighted average of the frequency by using the probability as a weight.

2. The system for detecting breathing according to claim 1, wherein
the transmission unit and the reception unit comprise a plurality of antennas.

3. The system for detecting breathing according to claim 1, wherein
the transmission unit further comprises a mechanism for changing an angle at which the radio waves are transmitted.

4. The system for detecting breathing according to claim 1, wherein
the generator generates the spectrogram by a Fourier transform with a window size of 15 to 50 seconds.

5. The system for detecting breathing according to claim 1, wherein
the radio waves are set at every 10° and a distance traveled by the radio waves is set at every 0.1 m.

6. The system for detecting breathing according to claim 1,
further comprising a parameter setter that sets a parameter for the breathing rate estimator by machine learning using the spectrogram.

7. The system for detecting breathing according to claim 6, wherein
the generator generates a person-present spectrogram for a case in which the subject is present and a person-absent spectrogram for a case in which the subject is absent, and
the machine learning is performed by using both of the person-present spectrogram and the person-absent spectrogram.

8. The system for detecting breathing according to claim 6, wherein
the generator generates a spectrogram for each orientation of the subject with respect to the transmission unit, and
the machine learning is performed based on the spectrogram.

9. The system for detecting breathing according to claim 6, wherein
the machine learning is performed by using a neural network.

10. The system for detecting breathing according to claim 9, wherein
in the neural network, processing of the spectrogram is performed in an order of convolution, dropout, convolution, pooling, dropout, convolution, dropout, convolution, and pooling.

11. A method for detecting breathing performed by a system for detecting breathing comprising:
a transmission unit that transmits radio waves to a plurality of positions including a position at which a subject is present; and
a reception unit that receives reflected waves as a result of reflection of the radio waves,
the method for detecting breathing comprising:
a phase fluctuation calculation step for calculating, by the system for detecting breathing, phase fluctuation of the reflected waves;
a generation step for performing, by the system for detecting breathing, a Fourier transform on the phase fluctuation and generating a spectrogram indicating a relationship between a time at which the reflected waves are received and a frequency component included in the reflected waves; and
a breathing rate estimation step for estimating, by the system for detecting breathing, a breathing rate of the subject by outputting a probability that the subject takes breaths at a predetermined frequency for each frequency based on the spectrogram and calculating a weighted average of the frequency by using the probability as a weight.
